(19) United States
(12) Patent Application Publication
(10) Pub. No.: US 2002/0168667 A1
(43) Pub. Date: Nov. 14, 2002

(54) Antigen detecting agent and antigen detecting kit, antigen detecting apparatus and antigen detecting method using the same

(75) Inventors: Takatoshi Kinoshita, Aichi (JP); Shintaro Washizu, Shizuoka (JP)

Correspondence Address: SUGHRUE MION, PLLC, 2100 Pennsylvania Avenue, NW, Washington, DC, 20037-3213, US

(73) Assignee: FUJI PHOTO FILM CO., LTD.

(21) Appl. No.: 10/103,831

(22) Filed: Mar. 25, 2002

(30) Foreign Application Priority Data
Mar. 23, 2001 (JP) 2001-86311

(51) Int. Cl. C12Q 1/70; C12Q 1/68; G01N 33/53; G01N 33/542; C12M 1/34

(52) U.S. Cl. 435/6; 435/5; 435/7.9; 435/287.2

(57) ABSTRACT

An antigen detecting agent having a rod-shaped body and an antibody bonded to the rod-shaped body which specifically bonds to a target antigen, an antigen detecting kit, an antigen detecting apparatus and an antigen detecting method using the antigen detecting agent are disclosed.

### BACKGROUND OF THE INVENTION

1. Field of the Invention

The present invention relates to an antigen detecting agent and also to an antigen detecting kit, an antigen detecting apparatus and an antigen detecting method using the same.

2. Description of the Related Art

For a detecting method utilizing an antigen-antibody reaction, radioimmunoassays (RIA), enzymatic immunoassays (EIA), fluorescent inmmunoassays (FIA), laser immunoassays (LIA), laser nephelometrys, FET immunoassays, and the like have been proposed, and some of them have been practically used currently.

Each of these methods are methods for detecting the presence or absence of an antigen to which isotopes, enzymes, fluorescent substances, and the like are added, respectively. However, in EIA methods, FIA methods and LIA methods, the sensitivity is 10⁻⁶ g or 10⁻¹⁰ g at best and in order to be an antigen test practically useful in an antigen-antibody reaction, their sensitivity was not high enough for practical use.

The RIA method has a sensitivity of 10⁻¹² g and is a measuring method which allows ultramicro analysis and antigen tests; however, since the method utilizes radioactive substances and accordingly a special device is required, there was a problem in terms of general use, cost, and the like.

As a method having a sensitivity of 10⁻¹² g or more in which an antigen test is possible and having a wide use, there have been proposed a laser magnetic immunoassay, and the like. However, this method also requires a special device and apparatus.

Thus, presently no antigen test agent and test method which fully satisfies sensitivity, wide use, operation, cost, and the like have been available and there has been a strong demand for such agents and methods.

### SUMMARY OF THE INVENTION

Thus, an object of the present invention is to provide an antigen detecting agent having a high sensitivity in which a target antigen may be easily and quickly detected and also to provide an antigen detecting kit, an antigen detecting apparatus and an antigen detecting method using the same.

The antigen detecting agent of the present invention has a rod-shaped body and an antibody which is bonded to the rod-shaped body and specifically bonds to a target antigen. Consequently, the target antigen may be surely detected by a simple operation.

The antigen detecting kit of the present invention contains an antigen detecting agent having a rod-shaped body of the length of 810 nm or shorter and an antibody which is bonded to the rod-shaped body and specifically bonds to a target antigen and reflects an incident light as colored interference light when aligned in a film-like shape; and any of dish, plate and tube.

The antigen detecting agent aligned in a film-like shape reflects the incident light as colored interference light on the basis of a multi-layer thin film interference theory which is a basic principle of color formation of scaly powder of the wings of a Morpho butterfly. When the change in wavelength based on the reflection of an incident light as colored interference light brought out by changes in length or refractive index at the time the antibody of the film-like antigen detecting agent bonds to a target antigen is measured, the target antigen in the specimen solution may be detected quickly by a simple operation in a reliable manner.

The first embodiment of an antigen detecting apparatus of the present invention utilizes an antigen detecting agent having a rod-shaped body of a length of 810 nm or shorter and an antibody which is bonded to the rod-shaped body and specifically bonds to a target antigen and reflects the incident light as colored interference light when aligned in a film-like shape; an adding means in which the antigen detecting agent contacts a sample, and a colored wavelength measuring means in which changes in the wavelength based on the reflection of the incident light as colored interference light of the antigen detecting agent which is bonded to the target antigen are measured.

The antigen detecting agent aligned in a film-like shape reflects the incident light as colored interference light on the basis of a multi-layer thin film interference theory which is a basic principle for color formation of scaly powder of the wings of a Morpho butterfly. When the change in wavelength based on the reflection of the incident light as colored interference light brought out by changes in length or refractive index at the time the antibody of the film-like antigen detecting agent bonds to target antigen is measured, presence of the target antigen may be detected.

The second embodiment of the antigen detecting apparatus of the present invention includes a biosensor having a rod-shaped body and an antibody which is bonded to the rod-shaped body and which specifically bonds to a target antigen in which an amphiphilic antigen detecting agent is adhered/bonded to a quartz oscillator or surface acoustic wave element in a film-like shape; an oscillation circuit in which changes in mass or changes in viscoelasticity when a target antigen is bonded to the biosensor are oscillated as a frequency; and a frequency counter whereby the frequency of the frequency oscillated from the oscillation circuit is measured.

As a result, changes in mass or changes in viscoelasticity when the antibody of the antigen detecting agent constituting the biosensor are subjected to an antigen-antibody reaction with a target antigen may be detected as a frequency with a high sensitivity and within a short time.

The antigen detecting method according to the present invention comprises a contacting step in which an antigen detecting agent having a rod-shaped body of a length of 810 nm or shorter, having an antibody which is bonded to the rod-shaped body and which specifically bonds to a target antibody and reflects the incident light as colored interference light when aligned in a film-like shape with a sample and a wavelength measuring step in which changes in wavelength based on the reflection of the incident light as colored interference light of the film-like antigen detecting agent bonded to the target antigen are measured.

The antigen detecting agent aligned in a film-like shape reflects the incident light as colored interference light on the basis of a multi-layer thin film interference theory which is a basic principle of color formation of scaly powder of the wings of a Morpho butterfly. When the change in wavelength based on the reflection of the incident light as colored interference light brought out by changes in length or refractive index at the time the antibody of the film-like antigen detecting agent bonds to a target antigen is measured, presence of the target antigen may be detected efficiently in a reliable manner.

### BRIEF DESCRIPTION OF THE DRAWINGS

FIG. 1 is a schematic view of a antigen detecting agent relating to one embodiment of the present invention.

FIG. 2 is a view for explaining a principle of light reflection of the incident light as colored interference light.

FIG. 3 is a typical view to explain the principle of light reflection of the incident light as colored interference light.

FIG. 4 is a schematic view for showing a formation of a monomolecular film by a functional molecule of the present invention.

FIG. 5 is a schematic view for showing an example of an amphiphilic functional molecule aligned on water (aqueous phase).

FIG. 6 is a schematic view for showing an example of an amphiphilic functional molecule vertically aligned on water (aqueous phase).

FIG. 7A and 7B are example views of a quartz oscillator in which FIG. 7A is a plan view and FIG. 7B is a front view.

FIG. 8 is a schematic view which shows an example of an antigen detecting apparatus.

FIG. 9 is a schematic plan view showing a surface acoustic wave (SAW) element.

### DESCRIPTION OF THE PREFERRED EMBODIMENTS

Herein after, the present invention will be described in detail.

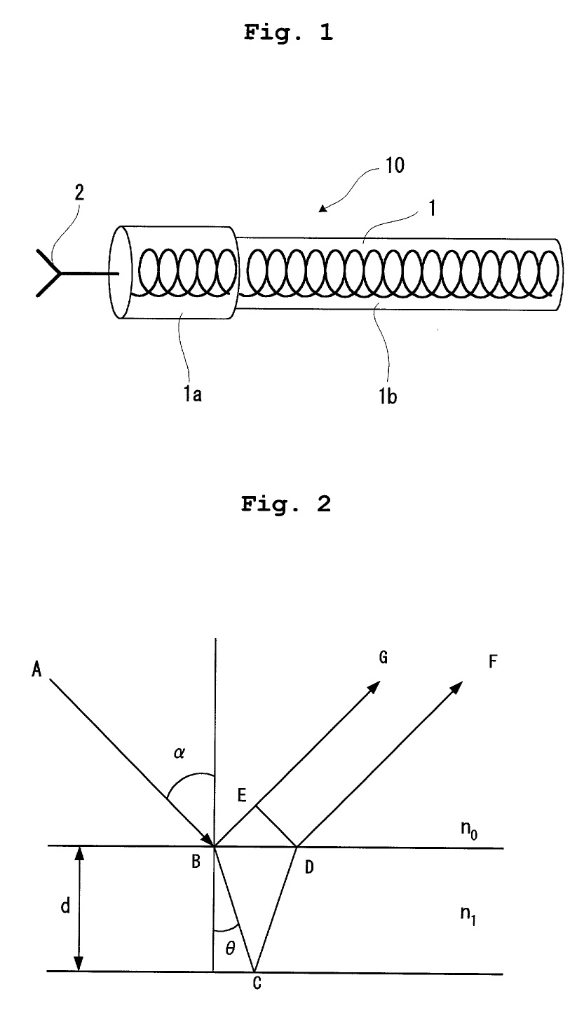

As shown in FIG. 1, the antigen detecting agent 10 of the present invention has a rod-shaped body 1 and an antibody 2 which is bonded to the rod-shaped body and which specifically bonds to a target antigen. Incidentally, the antigen detecting agent 10 of FIG. 1 is an amphiphilic polypeptide in an α-helix structure in which the rod-shaped 1a portion is hydrophobic while the 1b part is hydrophilic.

<Rod-Shaped Body>

The rod-shaped body is not particularly limited provided that it is rod-shaped, and may be appropriately selected in accordance with the object. The rod-shaped body may be either a rod-shaped inorganic substance or rod-shaped organic substance, but a rod-shaped organic substance is preferable.

Examples of rod-shaped organic substances are biopolymers, polysaccharides, and the like.

Suitable examples of biopolymers are fibrous proteins, α-helix polypeptides, nucleic acids (DNA, RNA), and the like. Examples of fibrous proteins are fibrous proteins having α-helix structures such as α-keratin, myosin, epidermin, fibrinogen, tropomyosin, silk fibroin, and the like. Suitable examples of polysaccharides are amylose and the like.

Among rod-shaped organic substances, spiral organic molecules whose molecules have a spiral structure are preferable from the standpoints of stable maintenance of the rod shape and internal intercalatability of other substances into the molecule in accordance with an object. Among the aforementioned substances, those with spiral organic molecules include α-helix polypeptides, DNA, amylose, and the like.

{α-helix Polypeptides}

α-helix polypeptides are referred to as one of the secondary structures of polypeptides. The polypeptide rotates one time (forms one spiral) for each amino acid 3.6 residue, and a hydrogen bond, which is substantially parallel to the axis of the helix, is formed between a carbonyl group (—CO—) and an imide group (—NH—) of each fourth amino acid, and this structure is repeated in units of seven amino acids. In this way, the α-helix polypeptide has a structure which is stable energy-wise.

The direction of the spiral of the α-helix polypeptide is not particularly limited, and may be either wound right or wound left. Note that, in nature, only structures whose direction of spiral is wound right exist from the standpoint of stability.

The amino acids which form the α-helix polypeptide are not particularly limited provided that an α-helix structure can be formed, and can be appropriately selected in accordance with the object. However, amino acids which facilitate formation of the α-helix structure are preferable. Suitable examples of such amino acids are aspartic acid (Asp), glutamic acid (Glu), arginine (Arg), lysine (Lys), histidine (His), asparagine (Asn), glutamine (Gln), serine (Ser), threonine (Thr), alanine (Ala), valine (Val), leucine (Leu), isoleucine (Ile), cysteine (Cys), methionine (Met), tyrosine (Tyr), phenylalanine (Phe), tryptophan (Trp), and the like. A single one of these amino acids may be used alone, or two or more may be used in combination.

By appropriately selecting the amino acid, the property of the α-helix polypeptide can be changed to any of hydrophilic, hydrophobic, and amphiphilic. In the case in which the α-helix polypeptide is to be made to be hydrophilic, suitable examples of the amino acid are serine (Ser), threonine (Thr), aspartic acid (Asp), glutamic acid (Glu), arginine (Arg), lysine (Lys), asparagine (Asn), glutamine (Gln), and the like. In the case in which the α-helix polypeptide is to be made to be hydrophobic, suitable examples of the amino acid are phenylalanine (Phe), tryptophan (Trp), isoleucine (Ile), tyrosine (Tyr), methionine (Met), leucine (Leu), valine (Val), and the like.

In the α-helix polypeptide, the carboxyl group, which does not form a peptide bond and which is in the amino acid which forms the α-helix, can be made to be hydrophobic by esterification. On the other hand, an esterified carboxyl group can be made to be hydrophilic by hydrolysis.

The amino acid may be any of a L-amino acid, a D-amino acid, a derivative in which the side chain portion of a L-amino acid or a D-amino acid is modified, and the like.

The number of bonds (the degree of polymerization) of the amino acid in the α-helix polypeptide is not particularly limited and may be appropriately selected in accordance with the object. However, 10 to 5000 is preferable.

If the number of bonds (the degree of polymerization) is less than 10, it may not be possible for the polyamino acid to form a stable α-helix. If the number of bonds (the degree of polymerization) exceeds 5000, vertical orientation may be difficult to achieve.

Suitable specific examples of the α-helix polypeptide are polyglutamic acid derivatives such as poly(γ-methyl L-glutamate), poly(γ-ethyl L-glutamate), poly(γ-benzyl L-glutamate), poly(n-hexyl L-glutamate), and the like; polyaspartic acid derivatives such as poly(β-benzyl L-aspartate) and the like; polypeptides such as poly(L-leucine), poly(L-alanine), poly(L-methionine), poly(L-phenylalanine), poly(L-lysine)-poly(γ-methyl L-glutamate), and the like.

The α-helix polypeptide may be a commercially available α-helix polypeptide, or may be appropriately synthesized or prepared in accordance with methods disclosed in known publications and the like.

As one example of synthesizing the α-helix polypeptide, the synthesis of block copolypeptide [poly(L-lysine)₂₅-poly(γ-methyl L-glutamate)₆₀]PLLZ₂₅-PMLG₆₀ is as follows. As is shown by the following formula, block copolypeptide [poly(L-lysine)₂₅-poly(γ-methyl L-glutamate)₆₀]PLLZ₂₅-PMLG₆₀ can be synthesized by polymerizing N^{ε}-carbobenzoxy L-lysine N^{α}-carboxy acid anhydride (LLZ-NCA) by using n-hexylamine as an initiator, and then polymerizing γ-methyl L-glutamate N-carboxy acid anhydride (MLG-NCA).

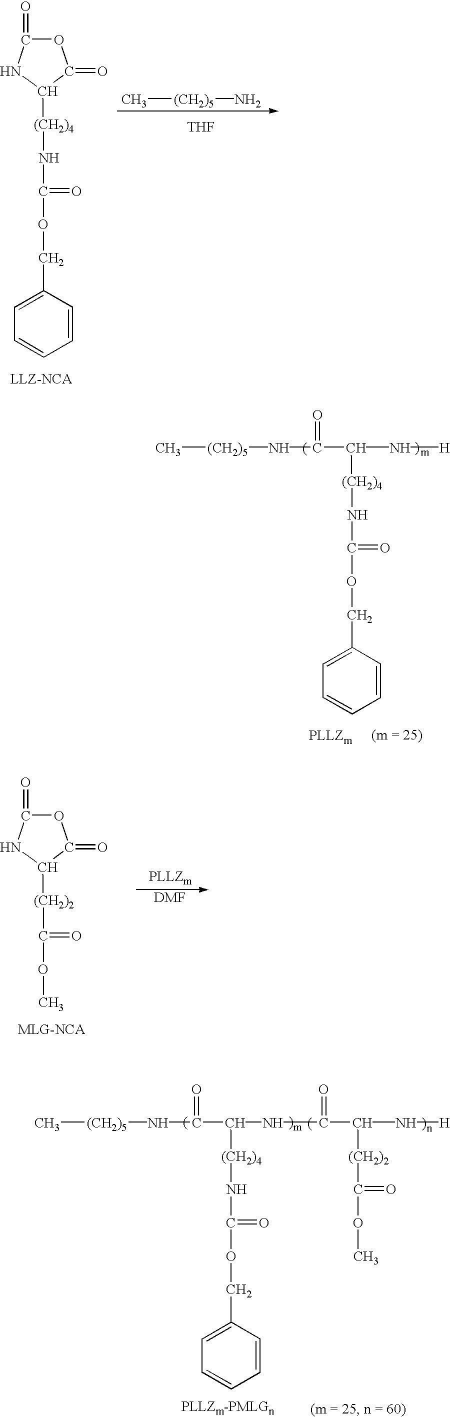

Synthesis of the α-helix polypeptide is not limited to the above-described method, and the α-helix polypeptide can be synthesized by a genetic engineering method. Specifically, the α-helix polypeptide can be manufactured by transforming a host cell by a expression vector in which is integrated a DNA which encodes the target polypeptide, and culturing the transformant, and the like.

Examples of the expression vector include a plasmid vector, a phage vector, a plasmid and phage chimeric vector, and the like.

Examples of the host cell include prokaryotic microorganisms such as E. coli, Bacillus subtilis, and the like; eukaryotic microorganisms such as yeast and the like; zooblasts, and the like.

The α-helix polypeptide may be prepared by removing the α-helix structural portion from a natural fibrous protein such as α-keratin, myosin, epidermin, fibrinogen, tropomyosin, silk fibroin, and the like. {DNA}

The DNA may be a single-stranded DNA. However, the DNA is preferably a double-stranded DNA from the standpoints that the rod-shape can be stably maintained, other substances can be intercalated into the interior of the molecule, and the like.

A double-stranded DNA has a double helix structure in which two polynucleotide chains, which are in the form of right-wound spirals, are formed so as to be positioned around a single central axis in a state in which they extend in respectively opposite directions.

The polynucleotide chains are formed by four types of nucleic acid bases which are adenine (A), thiamine (T), guanine (G), and cytosine (C). The nucleic acid bases in the polynucleotide chain exist in the form of projecting inwardly within a plane which is orthogonal to the central axis, and form so-called Watson-Crick base pairs. Thiamine specifically hydrogen bonds with adenine, and cytosine specifically hydrogen bonds with guanine. As a result, in a double-stranded DNA, the two polypeptide chains are bonded complementarily.

The DNA can be prepared by known methods such as PCR (Polymerase Chain Reaction), LCR (Ligase Chain Reaction), 3SR (Self-Sustained Sequence Replication), SDA (Strand Displacement Amplification), and the like. Among these, the PCR method is preferable.

Further, the DNA can be prepared by being directly removed enzymatically from a natural gene by a restriction enzyme. Or, the DNA can be prepared by a genetic cloning method, or by a chemical synthesis method.

In the case of a genetic cloning method, a large amount of the DNA can be prepared by, for example, integrating a structure, in which a normal nucleic acid has been amplified, into a vector which is selected from plasmid vectors, phage vectors, plasmid and phage chimeric vectors, and the like, and then introducing the vector into an arbitrary host in which propagation is possible and which is selected from prokaryotic microorganisms such as E. coli, Bacillus subtilis, and the like; eukaryotic microorganisms such as yeast and the like; zooblasts, and the like.

Examples of chemical synthesis methods include liquid phase methods or solid phase synthesis methods using an insoluble carrier, such as a tolyester method, a phosphorous acid method, and the like. In the case of a chemical synthesis method, the double-stranded DNA can be prepared by using a known automatic synthesizing device and the like to prepare a large amount of single-stranded DNA, and thereafter, carrying out annealing.

{Amylose}

Amylose is a polysaccharide having a spiral structure in which D-glucose, which forms starch which is a homopolysaccharide of higher plants for storage, is joined in a straight chain by α-1,4 bonds.

The molecular weight of the amylose is preferably around several thousand to 150,000 in number average molecular weight.

The amylose may be a commercially available amylose, or may be appropriately prepared in accordance with known methods.

Amylopectin may be contained in a portion of the amylose.

The length of the rod-shaped body is not particularly limited, and may be appropriately selected in accordance with the object. However, from the standpoint of causing light reflection of the incident light as colored interference light which will be described later, a length of 810 nm or less is preferable, and 10 nm to 810 nm is more preferable.

The diameter of the rod-shaped body is not particularly limited, and is about 0.8 to 2.0 nm in the case of the α-helix polypeptide.

The entire rod-shaped body may be hydrophobic or hydrophilic. Or, the rod-shaped body may be amphiphilic such that a portion thereof is hydrophobic or hydrophilic, and the other portion thereof exhibits the opposite property of the one portion. In the case of an amphiphilic rod-shaped body, the numbers of the lipophilic (hydrophobic) portions and hydrophilic portions are not particularly limited, and may be appropriately selected in accordance with the object. Further, in this case, the portions which are lipophilic (hydrophobic) and the portions which are hydrophilic may be positioned alternately, or either type of portion may be positioned only at one end portion of the rod-shaped body.

In the case of the amphiphilic rod-shaped body, there is no particular limitation for the numbers of the moiety showing hydrophobicity and the moiety showing hydrophilicity but that may be appropriately selected according to the object. In that case, the moiety showing hydrophobicity and the moiety showing hydrophilicity may be alternately positioned. Any of the moieties may be positioned only at one end of the rod-shaped body.

<Target Antigen>

The target antigen however, is not limited and may suitably be selected depending on an object, it is preferred to be at least one member selected from plasma protein, lipoprotein, glycoprotein, polypeptide, lipid, polysaccharide, lipopolysaccharide, nucleic acid and drug. It is particularly preferred to be plasma protein, tumor marker, apoprotein, viral antigen, autoantibody, coagulation/fibrinolysis factor, hormone, drug in blood or HLA antigen among them. It is not necessary that such a target antigen is an antigen which is the final target in the detection in each of the object as mentioned above but may be an antigen which co-exists with the antigen of the final target of detection.

Examples of the plasma protein include, immunoglobulin (IgG, IgA, IgM, IgD and IgE), complementary component (C3, C4, C5 and C1q), CRP, α₁-antitrypsin, α₁-microglobulin, β₂-microglobulin, haptoglobin, transferrin, celluloplasmin, ferritin, and the like.

Examples of the tumor marker include, α-fetoprotein (AFP), carcinoembryonic antigen (CEA), CA 19-9, CA 125, CA 15-3, SCC antigen, prostatic acidic phosphatase (PAP), PIVKA-II, γ-seminoprotein, TPA, elastase I, neuron-specific enolase (NSE), immunosuppressive acidic protein (IAP), and the like.

Examples of the apoprotein include, apo A-I, apo A-I, apo B, apo C-II, apo C-III, apo E, and the like.

Examples of the viral antigen include, antigen related to hepatitis B virus (HBV), antigen related to hepatitis C virus (HCV), HTLV-I, HIV, hydrophobia virus, influenza virus, rubella virus, and the like.

Examples of the HCV-related antigen include, HCVc100-3 recombinant antigen, pHCV-31 recombinant antigen, pHCV-34 recombinant antigen, and the like and a mixture thereof may be used preferably. Examples of the HIV-related antigen include virus surface antigen, and the like such as, for example, HIV-I env. gp 41 recombinant antigen, HIV-I env. gp 120 recombinant antigen, HIV-I gag. p 24 recombinant antigen, HIV-II env. p 36 recombinant antigen, and the like.

Examples of the infectious disease other than by virus include MRSA, ASO, toxoplasma, mycoplasma, STD, and the like.

Examples of the autoantibody include anti-microsome antibody, anti-thyroglobulin antibody, antinuclear antibody, rheumatism factor, anti-mitochondria antibody, myelin antibody, and the like.

Examples of the coagulation/fibrinolysis factor includes, fibrinogen, fibrin degradation product (FDP), plasminogen, α₂-plasmin inhibitor, antithrombin III, β-thromboglobulin, factor VIII, protein C, protein S, and the like.

Examples of the hormone include, pituitary hormone (LH, FSH, GH, ACTH, TSH and prolactin), thyroid hormone (T₃, T₄ and thyroglobulin), calcitonin, parathyroid hormone (PTH), adrenocortical hormone (aldosterone and cortisol), sex gland hormone (hCG, estrogen, testosterone and hPL), pancreaticogastrointestinal gland hormone (insulin, C-peptide, glucagon and gastrin) and others (renin, angiotensins I and II, enkephalin and erythropoietin).

Examples of the drug in blood include, antiepileptic drug such as carbamazepine, primidone and valproic acid; drugs for circulatory diseases such as digoxin, quinidine, digitoxin and theophylline; antibiotics such as gentamicin, kanamycin and streptomycin; and the like.

Examples of the sample to be examined containing the target antigen as such include pathogenic organisms such as bacteria and viruses; blood, saliva, disease tissue pieces, and the like separated from living organisms; and excrement such as feces and urine. Further, when diagnosis before birth is carried out, cells of a fetus existing in amniotic fluid and a part of divided ovules may be also used as a sample to be examined. Furthermore, such a sample to be examined may be used either directly or, if necessary, after concentrating as a precipitate by a centrifugal operation or the like and then subjected to a cytocidal treatment such as, for example, enzymatic treatment, thermal treatment, surfactant treatment, ultrasonic treatment or a combination thereof.

The antigen used in the present invention may also be that which is produced by a gene recombination method or is chemically synthesized on the basis of gene sequence or peptide sequence determined by gene recombination. Thus, it is a recombinant antigen which is prepared by such a manner that already-known genome sequence or DNA sequence obtained by a molecular cloning from natural virus or cell by utilization of gene recombination techniques is treated with enzymes or the like or subjected to chemical synthesis and the resulting DNA sequence or modified DNA sequence is expressed by a microbe, animal, plant, insect or the like to give a recombinant antigen or it is a peptide or a modified peptide which is prepared by means of a peptide chemical synthesis known as a liquid phase method or a solid phase method utilizing the above information. A solid phase synthetic method for peptide may be usually carried out by an automated peptide synthetic apparatus in an advantageous manner.

<Antibody Specifically Bonding to Target Antigen>

An antibody which is specifically bonded to the target antigen means that which specifically carries out an antigen-antibody reaction with the target antigen and it may be either a polyclonal antibody or a monoclonal antibody. It is also possible to use Fab′, Fab, F(ab′)₂, and the like of IgG, IgM, IgE and IgG.

The source of the antibody is not particularly limited and the antibody may be prepared by a conventional method. Thus, it may be prepared according to the methods described, for example, in Jikken Seibutsugaku Koza 14, Men-eki Seibutsugaku, edited by Shigeru Muramatsu, et al. (Maruzen Co., Ltd., 1985), Zoku Seikagaku Jikken Koza 5, Men-eki Seikagaku Kenkyuho, edited by Biochemical Society of Japan (Tokyo Kagaku Dojin, 1986), Shin Seikagaku Jikken Koza 12, Bunshi Men-ekigaku III, Kogen, Kotai, Hotai, edited by Biochemical Society of Japan (Tokyo Kagaku Dojin, 1992), and the like.

To be specific, antigen is administered to mammals such as horse, cattle, sheep, rabbit, goat, rat, mouse, and the like and the resulting immunized antiserum and ascites may be used per se or after purifying by conventionally known methods such as salting out (e.g., precipitating method with ammonium sulfate), gel filtration using Sephadex or the like, ion-exchange chromatographic method, electrophoretic method, dialysis, ultra filtration method, affinity chromatographic method, high-performance chromatographic method, and the like.

Further, when hybrid cells (hybridoma) are prepared from myeloma cells and spleen cells of mammals (such as mouse) immunized with antigen or the like and the resulting monoclonal antibody is used as a substance which is able to be specifically bonded to a specific component or, in case the specific component is a specific antibody, when its monoclonal antibody is modified and used as a mimetic specific component, that is preferred because of further improvement in the specificity, and the like.

The monoclonal antibody may be a monoclonal antibody which is prepared by utilizing a cell fusion technique using mouse myeloma cells disclosed, for example, by Kohler and Milstein (Kohler, G and Milstein, C., Nature, 256, 495 (1975)). The monoclonal antibody may be used after selecting from known ones and commercially available ones.

It is also possible to prepare the antibody by a gene recombination technique. With regard to such an antibody, each of the fractions such as IgG, IgM, IgA, IgE and IgD may be used. In addition, those enzymes may be treated with an enzyme such as trypsin, papain or pepsin and may be used as antibody fragment such as Fab, Fab′ or F(ab′)₂. Further, such an antibody may be used solely or plural antibodies may be used jointly.

When the resulting antibody specifically bonding to a target antigen is bonded to the rod-shaped body, an antigen detecting agent of the present invention is prepared.

The bonding method may be appropriately selected depending upon a material of the capturing structure and the rod-shaped body and there may be used known methods such as a method in which a covalent bond such as ester bond or amide bond is utilized, a method in which protein is labeled with avidin and is bonded to a biotinated capturing structure material, a method in which protein is labeled with streptoavidin and is bonded to a biotinated capturing structure material, and the like.

With regard to the covalent bond method, there may be exemplified peptide method, diazo method, alkylation method, cyan bromide activation method, bonding method by a cross-linking reagent, immobilization method utilizing Ugi reaction, immobilization method utilizing a thiol-disulfide exchange reaction, Schiff base formation method, chelate bonding method, tosyl chloride method, biochemically specific bonding method, and the like. For more stable bonding such as covalent bond, there is preferably carried out utilizing a reaction of thiol group with maleimide group, a reaction of pyridyl disulfide group with thiol group, a reaction of pyridyl disulfide group with thiol group, a reaction of amino group with aldehyde group, and the like and there may be applied a method which is appropriately selected from known methods, methods which may be easily carried out by the persons skilled in the art and methods which are modified therefrom. Among them, there may be used a chemically bonding agent and a cross-linking agent which are able to form more stable bond.

With regard to such chemically bonding agent and cross-linking agent, there may be exemplified carbodiimide, isocyanate, diazo compound, benzoquinone, aldehyde, periodic acid, maleimide compound, pyridyl disulfide compound, and the like. With regard to the preferred reagent, there may be exemplified glutaraldehyde, hexamethylene diisocyanate, hexamethylene diisothiocyanate, N,N′-polymethylenebisiodoacetamide, N,N′-ethylenebismaleiimide, ethylene glycol bissuccinimidyl succinate, bisdiazobenzidine, 1-ethyl-3-(3-dimethylaminopropyl) carbodiimide, succinimidyl 3-(2-pyridylthio) propionate (SPDP), N-succinimidyl 4-(N-maleimidomethyl) cyclohexane-1-carboxylate (SMCC), N-sulfosuccinimidyl 4-(N-maleimidomethyl) cyclohexane-1-carboxylate, N-succinimidyl (4-iodoacetyl) aminobenzoate, N-succinimidyl 4(1-maleimidophenyl) butyrate, iminothiolane, S-acetylmercaptosuccinic acid anhydride, methyl-3-(4′dithiopyridyl) propionimidate, methyl-4-mercaptobutyryl imidate, methyl-3-mercaptopropionimidate, N-succinimidyl-S-acetyl mercaptoacetate, and the like.

<Antigen Detecting Agent>

As shown in FIG. 1, the antigen detecting agent of the present invention has a rod-shaped body 1 and an antibody 2 which is bonded to the rod-shaped body 1 and which specifically bonds to a target antigen. In the antigen detecting agent, when the target antigen is bonded to the antibody part, properties of the antigen detecting agent such as refractive index and transmittance of light, mass, viscoelasticity, and the like change.

Thus, when the change is detected, that may be utilized for the detection of antigen.

The above method for the detection may be appropriately selected according to the object and, for example, various methods such as that color change is observed by naked eye, that wavelength change is detected by spectrophotometer, that oscillation of frequency of quartz oscillator, surface acoustic wave (SAW) element and the like is detected by a frequency counter, and the like may be carried out.

The antigen detecting agent may be used alone, and in that case, when it is used by aligning in single or plural layer(s) on the surface of a solvent containing the subject to be captured or at the boundary between the solvent and a liquid having a reverse affinity to the solvent is preferred, since changes in wavelength may easily be detected.

It is also possible to form in a film like manner such as monomolecular film or two layered monomolecular films on a substrate which is vertically aligned by, for example, a Langmuir-Brodgett (LB) technique.

With regard to the antigen detecting agent of the present invention, the one which is able to reflect the incident light as colored interference light is preferred from a viewpoint of recognition and discrimination.

The reflection of the incident light as colored interference light is a color formation on the basis of a multi-layer thin film interference theory which is a basic principle for color formation of scaly powder of the wings of a Morpho butterfly and is a color formation on the film as a result of the reflection of the light of specific wavelength corresponding to the thickness of the film and the refractivity thereof when stimulation from outside such as electric field, magnetic field, heat, light (for example, natural light, infrared light and ultraviolet light), and the like is applied to the film. The color tone may be freely controlled like the surface skin of a chameleon by the stimulation from outside.

The principle of light reflection of the incident light as colored interference light will be described herein after.

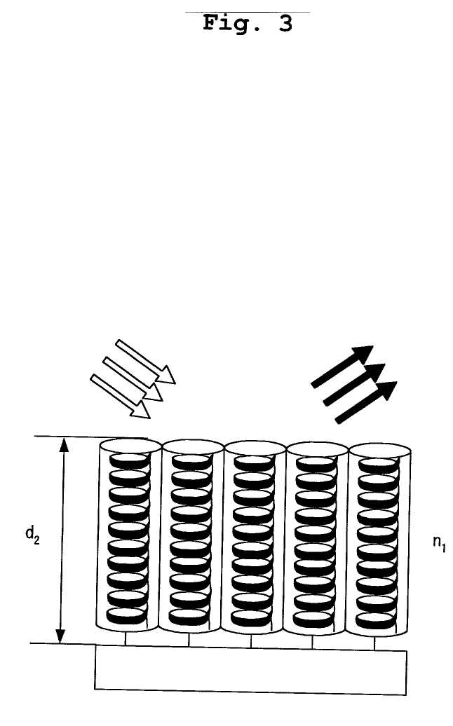

As shown in FIG. 2 and FIG. 3, when light is irradiated on the film of the rod-shaped body, the wavelength (λ) of the interference light by the film is emphasized under the condition as shown in the following (1) and enfeebled under the condition as shown in the following (2).

$$\begin{matrix}λ = \frac{2 tl}{m} \sqrt{{n}^{2} - {sin}^{2} α} & ( 1 ) \\ λ = \frac{4 tl}{2 m - 1} \sqrt{{n}^{2} - {sin}^{2} α} & ( 2 )\end{matrix}$$

In the formulae (1) and (2), λ means wavelength (nm) of the interference light, α means angle of incidence (degree) of the light to the film, t means thickness (nm) of a single film, 1 means the number of layers of the film, n means the refractive index of the film and m means an integer of 1 or more.

The light reflection of the incident light as colored interference light is available by aligning the antigen detecting agent in a film-like shape.

Thickness of the single film is preferably 810 nm or less and, more preferably, it is from 10 nm to 810 nm.

When the thickness is suitably changed, a color (wavelength) of the interference light by the light reflection of the incident light may also be changed.

The film may be either a monomolecular film or a layered film comprising the monomolecular film.

The monomolecular film or the layered film comprising the same may be formed by, for example, a Langmuir-Brodgett method (LB method) and, in that case, a known LB film forming apparatus (such as NL-LB 400 NK-MWC manufactured by Nippon Laser & Electronics Laboratories) may be used.

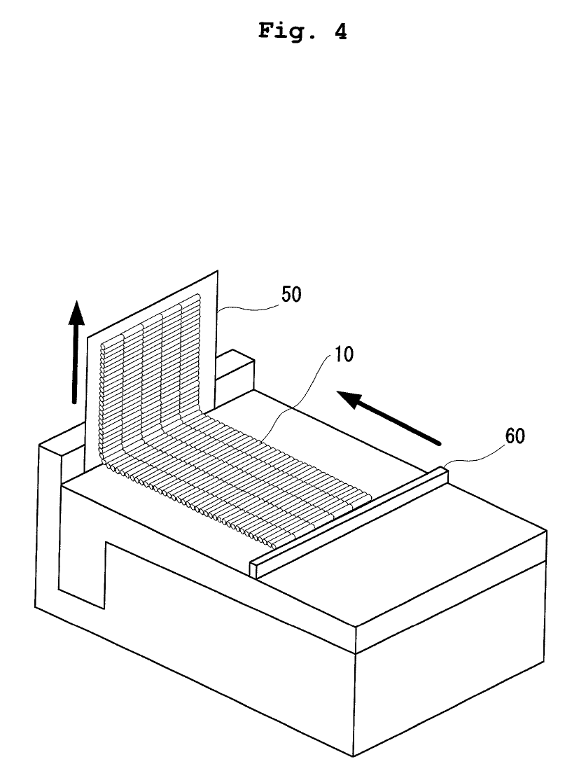

Formation of the monomolecular film may be carried out, for example, in such a state that the above mentioned rod-shaped body which is lipophilic (hydrophobic) or amphiphilic is floated on water surface (on an aqueous phase) or in such a state that the rod-shaped body which is lipophilic (hydrophobic) or amphiphilic is floated on oil surface (on an oil phase) or, in other words, the rod-shaped body 1 is aligned as shown in FIG. 4 so as to form on a substrate 50 using an extrusion material 60. When such an operation is repeatedly carried out, the layered film where the monomolecular films are layered in any number may be formed on the substrate 50. Incidentally, it is preferred that the monomolecular film or the layered film is fixed on the substrate 50 since the reflection of the incident light as colored interference light by the monomolecular film or layered film is expressed in a stable manner.

In that case, there is no particular limitation for the substrate 50 and, according to the object, its material, shape, size, and the like may be appropriately selected although it is preferred that its surface is appropriately subjected to a surface treatment previously with an object that the rod-shaped body 1 is easily adhered or bonded thereto. When the rod-shaped body 1 (such as cc-helix polypeptide) is hydrophilic for example, it is preferred that a surface treatment such as hydrophilizing treatment using octadecyl trimethylsiloxane and the like is previously carried out.

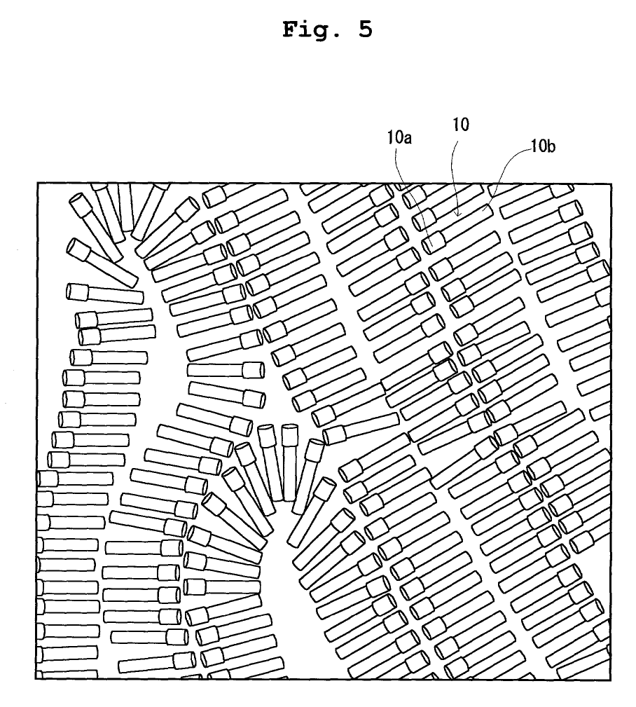

With regard to the state where the rod-shaped body is floated on an oil phase or an aqueous phase in the formation of the monomolecular film of the amphiphilic rod-shaped body, the lipophilic areas (hydrophobic areas) 1a of the rod-shaped body 1 are aligned in an adjacent state to each other on the aqueous phase or oil phase while the hydrophilic areas 1b are aligned in an adjacent state to each other as shown in FIG. 5.

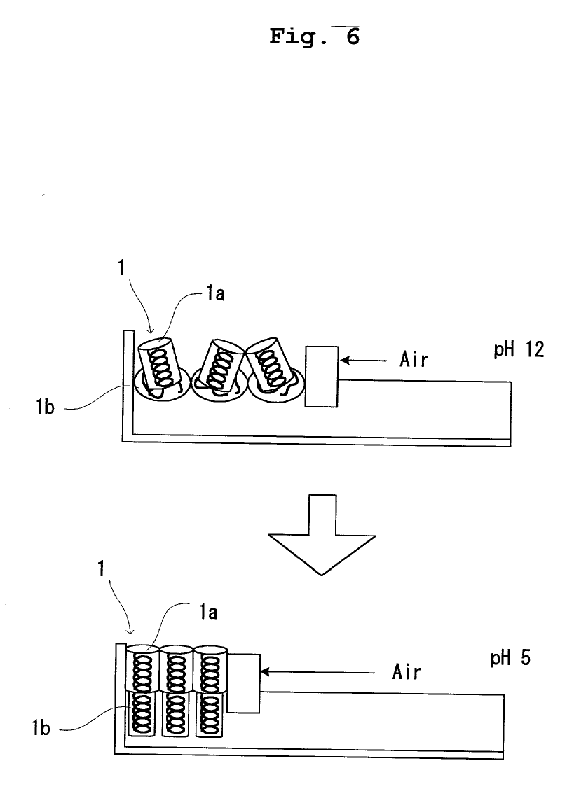

The above is an example of a layered membrane or a layered film comprising the same where the rod-shaped body is aligned in the plane direction of the monomolecular film (in a horizontal state) while a monomolecular film where the rod-shaped body is aligned in the thickness direction of the monomolecular film (in a vertical state) may be manufactured, for example, as follows. First, as shown in FIG. 6, water (aqueous phase) is made alkaline of around pH 12 under such a state that the amphiphilic rod-shaped body 1 (α-helix polypeptide) is floated on the water surface (aqueous phase) (i.e., in a horizontal state). As a result, in the hydrophilic area 1b in the rod-shaped body 1 (α-helix polypeptide), the α-helix structure thereof is disentangled to give a random structure. At that time, the lipophilic area (hydrophobic area) 1a of the rod-shaped body 1 (α-helix polypeptide) maintains its α-helix structure. Then, the pH of the water (aqueous phase) is made acidic to about 5 thereby the hydrophilic area 1b in the rod-shaped body 1 (α-helix polypeptide) forms an α-helix structure again. When the pushing material attached to the rod-shaped body 1 (α-helix polypeptide) is pushed by the pressure of air from its side to the rod-shaped body 1 (α-helix polypeptide), the rod-shaped body 1 maintains vertical against water (aqueous phase) while its hydrophilic area 1b forms an α-helix structure in the direction substantially orthogonal to the water surface in the aqueous phase. When the aligned rod-shaped body 1 (α-helix polypeptide) is pushed out onto the substrate 50 using a pushing material 60 as mentioned above by referring to FIG. 4, it is possible to form a monomolecular film on the substrate 50. When such operation is repeatedly carried out, the layered film having a prescribed number of monomolecular films may be formed on the substrate 50.

With regard to the antigen detecting agent which is able to provide a single-layered film or multi-layered film reflecting an incident light as a colored interference light, there may be exemplified an antigen detecting agent which is amphiphilic and an amphiphilic antigen detecting agent wherein the rod-shaped body is α-helix polypeptide is preferred.

The antigen detecting agent of the present invention may be that which shows a sedimentation reaction by a specific bonding to the target antigen.

The antigen-antibody reaction using the antigen detecting agent of the present invention may be carried out in such a manner that antigen and antibody are added so as to make their ratio in a physiological saline optimum and then a reaction is carried out under the condition of pH 6-8 at around 37° C.

<Antigen Detecting Kit>

The antigen detecting kit of the present invention contains an antigen detecting agent having a rod-shaped body of the length of 810 nm or shorter and an antibody which is bonded to the rod-shaped body and which specifically bonds to a target antigen and reflects the incident light as colored interference light when aligned in a film-like shape; and any of a dish, a plate and a tube.

The antigen detecting kit may contain a solvent containing the antigen detecting agent in an amount suitable for the size of the dish, and the like in a container which is different from the container. For example, an aqueous specimen solution is added to the container and the oily or amphiphilic antigen detecting agent is added to the specimen solution so that the antigen detecting agent is aligned on the sample in a film-like shape whereby a target antigen may be detected by the changes in wavelength caused by the reflection of the incident light as a colored interference light of the film-like antigen detecting agent.

If necessary, the detecting kit may be combined with a reagent for pretreatment of the sample, a washing liquid, oil for preventing the evaporation of water from the reaction solution, and the like.

In the nucleic acid detecting kit of the present invention, the antigen detecting reagent aligned in a film-like shape reflects the incident light as colored interference light on the basis of the multi-layered thin film interference theory which is a basic principle for coloration of scaly powder of the wings of a Morpho butterfly. Accordingly, when the changes in wavelength caused by the reflection of the incident light as a colored interference light brought out by changes in refractive index or length at the time an antigen-antibody of the film-like antigen detecting reagent reacts with the target antigen are measured, it is now possible to detect the target antigen in the specimen solution by a simple operation in a reliable manner.

<Antigen Detecting Apparatus>

The antigen detecting apparatus according to the first embodiment of the present invention is equipped with an antigen detecting agent having a rod-shaped body of a length of 810 nm or shorter, an antibody which is bonded to the rod-shaped body and which specifically bonds to a target antigen and reflects the incident light as colored interference light when aligned in a film-like shape; an adding means in which the antigen detecting agent is contacted to a specimen solution; and a colored wavelength measuring means in which changes in wavelength caused by the light reflection of the incident light as colored interference light of the film-like antigen detecting agent which is bonded to the target antigen are measured.

The sample is not particularly limited so long as the sample may be an object for detecting whether it contains a target antigen or not, and for instance, a solution may be mentioned.

With regard to the adding means, there is no particular limitation so long as it is a means for adding a predetermined amount of the antigen detecting agent to the specimen solution or is a means for adding a predetermined amount of the specimen solution to the antigen detecting agent. It is however preferred that the amount of the antigen detecting agent is determined in such an amount that the reflection of the incident light as interference light may apt to be detected by aligned in a film-like shape.

In that case, when an antibody which specifically bonds to the target antigen in the antigen detecting agent is subjected to an antigen-antibody reaction with the target antigen, the refractive index or length of the antigen detecting agent change and, therefore, when the change in wavelength is measured by a colored wavelength measuring means such as a spectrophotometer, it is possible to specifically test the presence or absence of the target antigen. When a calibration curve is previously prepared using a known amount of sample antigen, the concentration of the antigen to be detected or quantified in the specimen solution may be detected or quantified.

One of the preferred embodiments of the antigen detecting apparatus is an antigen detecting apparatus in which the antigen detecting agent is amphiphilic and the adding means is an adding means in which the antigen detecting agent is added to an aqueous specimen solution together with an oil phase so that the antigen detecting agent and the sample are contacted.

In that case, the antigen detecting agent is amphiphilic and, therefore, it is preferred because the antigen detecting agent is vertically aligned at the interface of an oil phase and an aqueous phase to form a film and changes in wavelength brought out by the reflection of the incident light as interference light are easily measured.

The antigen detecting apparatus in accordance with the second aspect of the present invention is that it is provided with a biosensor where the antigen detecting agent of the present invention is adhered and bonded in a film-like shape to a quartz oscillator or a surface acoustic wave (SAW) element, an oscillation circuit where changes in mass or changes in viscoelasticity when the object to be captured is captured by the biosensor are oscillated as a frequency and a frequency counter where the frequency of the oscillation oscillated from the oscillation circuit is measured.

In that case, it is preferred that the antigen detecting agent is adhered and bonded in a monomolecular film-like shape to the quartz oscillator or to the surface acoustic wave (SAW) element or is adhered and bonded in a two layered monomolecular film-like shape thereto. With regard to the frequency counter, there is no particular limitation so far as it is able to precisely measure the frequency from the quartz oscillator or the surface acoustic wave (SAW) element.

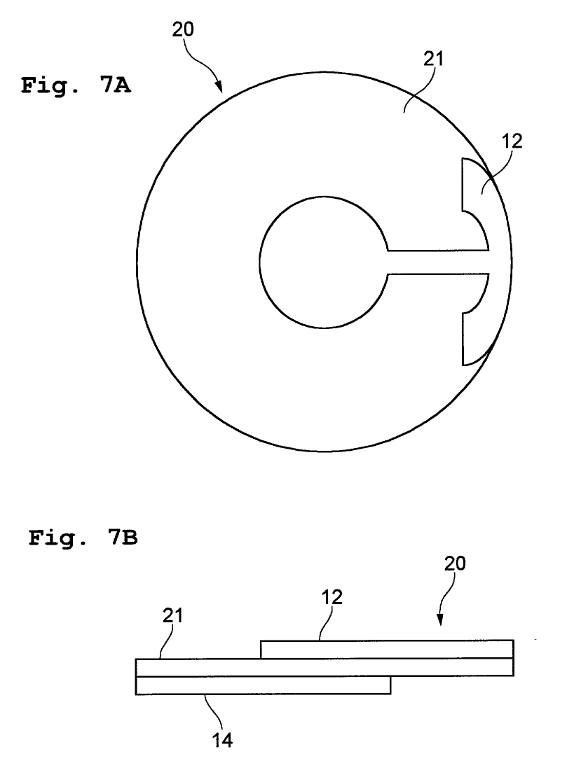

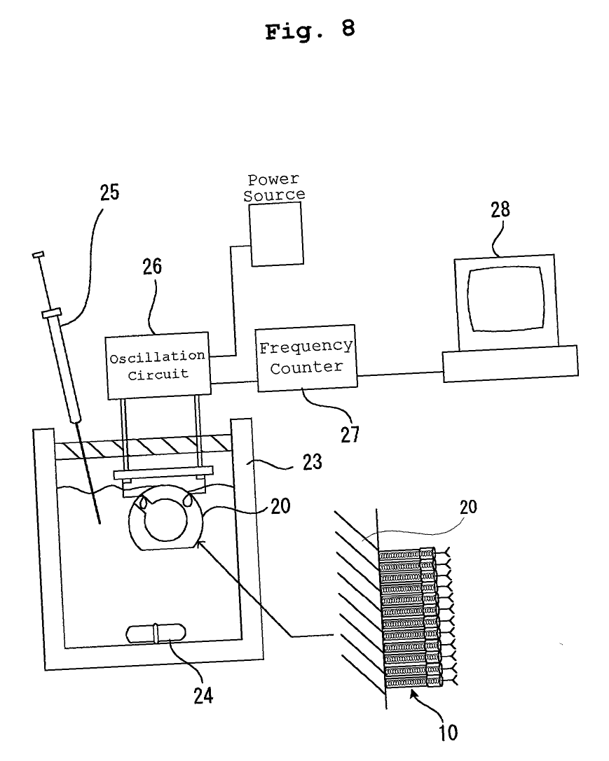

In the quartz oscillator, metal electrodes are vapor deposited on the surface and the back of a thin quartz plate. An example of the quartz oscillator 20 is shown in FIG. 7A and 7B. FIG. 7A is a plane view while FIG. 7B is a front view. An electrode 12 is vapor deposited on the surface of the quartz plate 21 while another electrode 14 is vapor deposited on the back thereof. The electrodes extend to the left side from the electrodes 12, 14 and the left ends thereof are connected to clip-type lead wires (not shown) followed by connecting to an alternating current source (not shown in the drawings). When alternating current is applied between the electrodes 12, 14, there is generated oscillation of a predetermined period in the quartz plate 21 due to a back piezoelectric effect.

Although not shown in the drawing, an antigen detecting agent film is adhered/bonded to the surface of the quartz oscillator 20. The antibody of this antigen detecting agent film is bonded to the target antigen and mass of the surface of the quartz oscillator 20 changes to an extent of the mass of the bonded target antigen whereby a resonance frequency changes.

Between the changes in the resonance frequency and changes in the mass of the antigen detecting agent film coated on the surface of the quartz oscillator 20 which oscillates in parallel to the plane vertical to the thickness direction, there is a relation as shown in the following formula (3) whereby changes in the mass may be detected from changes in the resonance frequency. For example, in the case of an oscillator of resonance frequency of 9 MHz (area: about 0.5 cm²), a reduction in frequency of 400 Hz is resulted by an increase in mass of 1 μg.

ΔF=−2.3×10⁶(F²×ΔW/A) (3)

In the formula, F means resonance frequency (MHz) of the quartz oscillator, ΔF means changes (Hz) in the resonance frequency by changes in mass, ΔW means changes in mass (g) of the film and A means the surface area (cm²) of the film.

An example of the antigen detecting apparatus is shown in FIG. 8. The quartz oscillator 20 (antigen detecting agent 10 is bonded on the surface in a film-like shape) is attached to an arm for attaching the quartz oscillator and dipped in a solution in a thermostat heat block 23. The thermostat heat block 23 maintains the temperature of the solution constant. The solution is stirred by a stirrer 24. In a sample injection 25, a sample to be measured is injected into a solution. In the oscillation circuit 26, alternating current field is applied to the electrodes 12, 14 of the quartz oscillator 20 to oscillate the quartz oscillator 20. Oscillation frequency of the oscillation circuit 24 is counted by a counter 27, analyzed by a computer 28 and the mass of the target antigen in the specimen solution is indicated.

The antibody in the antigen detecting agent is subjected to an antigen-antibody reaction with the target antigen in such a way in which the mass of the antigen detecting agent changes. The change in the mass is caught or reflected by the quartz oscillator and converted to frequency and, therefore, when the change in frequency is measured by the frequency counter, the presence or absence of the target antigen may be specifically tested.

When a calibration curve is previously prepared using an object to be captured of a known amount, the object to be captured concentration to be detected or quantified in the specimen solution may be detected or quantified.

The surface acoustic wave (SAW) element is an element where a pair of comb-shaped electrodes is set on the surface of a solid and an electric signal is converted to a surface acoustic wave (sonic wave transmitting the solid surface, ultrasonic wave), transmitted to the encountering electrode and outputted as an electric signal again whereby a signal of specific frequency corresponding to the stimulation may be taken out. Ferroelectric a substance such as lithium tantalite and lithium niobate, quartz, zinc oxide thin film, and the like are used as the material therefor.

The SAW is an elastic wave which transmits along the surface of the medium and exponentially decreases in the internal area of the medium. In the SAW, the transmitted energy is concentrated on the surface of the medium whereby the changes in the medium surface may be sensitively detected and, as a result of the changes in the mass of the surface, the SAW transmitting velocity changes the same as in the case of a quartz oscillator. Usually, SAW transmitting velocity is measured as the changes in oscillation frequency using an oscillation circuit. Changes in the oscillation frequency are given by the following formula.

Δf=(k₁+k₂)f²−hρ−k₂f²h[(4μ/Vᵣ²)(λ+μ/λ+2μ)]

In the formula, k₁ and k₂ mean constants, h means thickness of the fixed film, ρ means density of the film, λ and μ mean Lame constants of the film and Vᵣ means a SAW transmitting velocity.

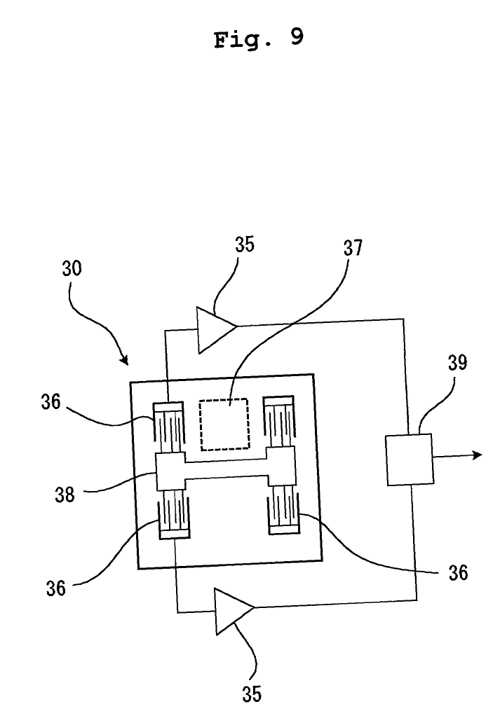

FIG. 9 is a schematic plane view which shows an example of constitution of main parts of a surface acoustic wave (SAW) element. In FIG. 9, in the SAW element sensor 30, there are formed gold electrode 38 and comb-shaped electrodes 36 at both ends thereof on the SAW element having a resonance frequency of 90 MHz made of an ST cut quartz and there is formed a film (not shown) comprising the antigen detecting agent in the surface wave transmitting region 37 as shown by dotted lines. The sensor is connected to a frequency counter 39 from each comb-shaped electrode 36 via a high-frequency amplifier 35 whereby the mass of the object to be captured in the specimen solution is indicated.

When the antibody which specifically bonds to the target antigen in the antigen detecting agent is subjected to an antigen-antibody reaction with the target antigen, the mass or viscoelasticity of the antigen detecting agent changes and the mass change or viscoelasticity change is detected by the surface acoustic wave (SAW) element and converted to a frequency. Therefore, when this frequency change is measured by the frequency counter, it is now possible to specifically examine whether or not the target antigen is present.

When a calibration curve is previously prepared using a sample antigen of a known amount, the antigen concentration to be detected or quantified in the specimen solution may be detected or quantified.

With regard to a method for a chemical bonding/fixing of the antigen detecting agent on the electrodes of the quartz oscillator or the surface acoustic wave (SAW) element which constitutes the biosensor, there is no particular limitation and may be appropriately selected depending on the object. For example, it may be carried out by means of a chemical bond such as covalent bond.

With regard to the covalent bond method, there is no particular limitation but the same one which is used for bonding the rod-shaped body and the antibody in the antigen detecting agent may be appropriately selected and used.

To be specific, for example, a method in which a substance where thiol group is introduced into the end of the antigen detecting agent is synthesized, the quartz oscillator or the surface acoustic wave (SAW) element is dipped in its solution and made to react therewith for a predetermined time and then the biosensor is taken out from the solution thereafter drying. The thiol group covers S-trityl-3-mercaptopropyloxy-β-cyanoethyl-N,N-diisopropyl-amino phosphoramidide and the like and introduction of the thiol group into the end of the antigen testing may be carried out by a phosphoramidide method.

<Antigen Detecting Method>

The antigen detecting method according to the present invention comprises a contacting step in which an antigen detecting agent having a rod-shaped body of a length of 810 nm or shorter, having an antibody which is bonded to the rod-shaped body and which specifically bonds to a target antibody and reflects the incident light as colored interference light when aligned in a film-like shape with a sample; and a wavelength measuring step in which changes in wavelength brought out by light reflection of the incident light as colored interference light of the film-like antigen detecting agent bonded to the target antigen are measured.

With regard to the colored wavelength measuring means, there is no particular limitation so long as it is a method in which changes in the wavelength on the basis of the light reflection of the incident light as colored interference light by changes in refractive index or length on an antigen-antibody reaction of the antibody of the antigen detecting agent aligned in a film-like shape with the target antigen may be measured and examples include a method in which the wavelength change is measured using a spectrophotometer.

In accordance with the antigen detecting method of the present invention, the wavelength change on the basis of the light reflection of the incident light as colored interference light by the change in refractive index or length when antigen-antibody of the antibody in the antigen detecting agent reacts with the target antigen is measured in which the presence of the target antigen may be quickly detected by a simple operation in a reliable manner.

### EXAMPLES

Hereinafter, the present invention will be described using examples although the present invention should not be limited by the examples.

### Example 1

Polymerization of N^{ε}-carbobenzoxy L-lysine N^{α}-carboxylic acid anhydride (LLZ-NCA) was carried out using n-hexylamine as an initiator and then polymerization of γ-methyl L-glutamate N-carboxylic acid anhydride (MLG-NCA) was carried out to prepare a block copolypeptide PLLZ₂₀₀₀-PMLG₆₀₀ in which degree of polymerization of a PLLZ moiety was 2000 and that of a PMLG moiety was 600. After that, the PMLG segment was partially hydrolyzed to give L-glutamic add (LGA) and an α-helix copolypeptide PLLZ₂₅₀-P(MLG₄₂₀/LGA₁₈₀) was prepared.

Avidin was introduced into this α-helix copolypeptide and a biotin-labeled anti-hepatitis B antigen IgG is bonded thereto via a biotin-avidin bond to prepare an antigen detecting agent.

After that, the antigen detecting agent was floatedly (i.e., in a horizontal state) placed on the surface of water (aqueous phase) and the pH of the water (aqueous phase) was made alkaline of approximately 12. Incidentally, the α-helix structure in the hydrophilic moiety in the antigen detecting agent was disentangled to form a random structure, during in which the hydrophobic moiety of the antigen detecting agent maintained its α-helix structure. After that, the pH of the water (aqueous phase) was made acidic to approximately 5. Consequently, the hydrophilic moiety of the antigen detecting agent was made into the α-helix structure again. At that time, when the pushing material attached to the antigen detecting agent was pushed from the side thereof by the pressure of air to the antigen detecting agent, the hydrophilic moiety was made into the α-helix structure in the direction orthogonal against the surface of water in the aqueous phase while the antigen detecting agent maintained vertical state against the water (aqueous phase). Then, as mentioned, when the antigen detecting agent in an aligned state was pushed onto the substrate (plate) using the pushing material, it was possible to form a monomolecular film in which the antigen detecting agent was vertically stood on the substrate (plate). Incidentally, the above operation was carried out using an LB film forming apparatus (NL-LB 400 NK-MWC; manufactured by Nippon Laser & Electronics Laboratories). Thickness of this monomolecular film was calculated to be about 16 nm.

The substrate in which the monomolecular film comprising the antigen detecting agent vertically disposed was added to a specimen solution which has positive HBs and changes in wavelength caused by the reflection of the incident light as a colored interference light were measured using a spectrophotometer, a significant change in the wavelength in the polypeptide was observed as compared to an antigen detecting agent without bonding anti-hepatitis B antigen IgG.

### Example 2

The monomolecular film in which the antigen detecting agent was vertically formed on the substrate (plate) in Example 1 was used as a constituting unit comprising two layers to prepare a substrate in which the antigen detecting agent was vertically disposed in a two layered monomolecular films form. This substrate was placed in a specimen solution which has positive HBs and changes in wavelength caused by the reflection of the incident light as a colored interference light were measured by a spectrophotometer in which a significant change in the wavelength was observed as compared to the antigen detecting agent without bonding anti-hepatitis B surface antigen IgG.

### Example 3

A product in which a gold electrode having an area of 0.2 cm² and a gold-plated lead wire attached to a quartz oscillator (AT cut; area: 0.5 cm²; basic frequency: 9 MHz) was used as a quartz oscillation electrode.

The quartz oscillation electrode was dipped at room temperature for 1 hour in a 1% by volume aqueous solution of aminopropyl triethoxysilane (manufactured by Chisso) and washed by irradiation of ultrasonic waves of 20 kHz in pure water for 30 minutes to remove excess aminopropyl triethoxysilane. Then, the quartz oscillation electrode was subjected to a thermal treatment for 20 minutes at the temperature of 110° C. whereby a covalent bond was formed between aminopropyl triethoxysilane and the quartz oscillator surface.

The quartz oscillator was dipped for 1 hour in a 1% by volume aqueous solution of glutaraldehyde to form a covalent bond between glutaraldehyde and aminopropyl triethoxysilane and, nextly, the quartz oscillator was washed by irradiation with ultrasonic waves of 20 kHz for 30 minutes in pure water to remove excess glutaraldehyde.

The quartz oscillator electrode was dipped for 2 hours in 100 ml of phosphate buffer of pH 7.2 containing the antigen detecting agent prepared in Example 1. Consequently, the antigen detecting agent was fixed to the quartz oscillator via glutaraldehyde. Then, unreacted antigen detecting agent was removed by washing with a phosphate buffer of pH 7.2.

After that, the quartz oscillator prepared as such was attached to the antigen detecting apparatus as shown in FIG. 8, a predetermined amount of a solution to be tested which has positive HBs was added and the changes in the frequency in 10 minutes were checked. Within one minute, changes in the oscillation frequency nearly reached saturation. The thing to which an HBs-positive solution to be tested was added showed a significant reduction in oscillation frequency as compared to that to which no such a solution was added.

It was also observed that, when the added amount of the HBs-positive solution to be tested was increased, the oscillation frequency decreased in a certain rate.

### Example 4

An antigen detecting apparatus was assembled by the same manner as in Example 3 except that a surface acoustic wave (SAW) element of ST cut having oscillation frequency of 10.3 MHz as shown in FIG. 9 was used in place of the quartz oscillator in Example 3.

A predetermined amount of an HBs-positive solution to be tested was added thereto and the changes in frequency during 10 minutes were checked. Within one minute, the changes in the oscillation frequency nearly reached saturation. A sample to which an HBs-positive solution to be tested was added showed a significant reduction in oscillation frequency as compared to the one without a solution added.

It was also observed that, when the added amount of the HBs-positive solution to be tested was increased, the oscillation frequency decreased in a certain rate.

In accordance with the present invention, shortcomings such as time consuming measurements, and special device requirements of the prior art may be avoided, and various types of target antigens are efficiently tested in an aqueous phase or an oil phase under high sensitivity in a simple and quick manner.

What is claimed is:

1. An antigen detecting agent comprising:
a rod-shaped body; and
an antibody bonded to the rod-shaped body and which specifically bonds to a target antigen.

2. An antigen detecting agent according to claim 1, wherein the target antigen is at least one antigen selected from the groups consisting of proteins, lipoproteins, glycoproteins, polypeptides, lipids, polysaccharides, lipopolysaccharides, nucleic acids and drugs.

3. An antigen detecting agent according to claim 2, wherein the target antigen is at least one antigen selected from the group consisting of plasma proteins, tumor markers, apoproteins, viruses, autoantibodies, coagulation/fibrinolysis factors, hormones, drugs in blood and HLA antigens.

4. An antigen detecting agent according to claim 1, wherein the target antigen is an antigen which coexists with an antigen of the final target.

5. An antigen detecting agent according to claim 1, wherein the agent exhibits a sedimentation reaction by specifically bonding to the target antigen.

6. An antigen detecting agent according to claim 1, wherein the antigen detecting agent is amphiphilic.

7. An antigen detecting agent according to claim 1, wherein the rod-shaped body is a helical organic molecule.

8. An antigen detecting agent according to claim 7, wherein the helical organic molecule is any of α-helix polypeptide, DNA and amylose.

9. An antigen detecting agent according to claim 8, wherein the antigen detecting agent is amphiphilic.

10. An antigen detecting agent according to claim 9, wherein the helical organic molecule is a block polymer of α-helix polypeptide.

11. An antigen detecting agent according to claim 1, wherein a length of the rod-shaped body is 810 nm or shorter.

12. An antigen detecting agent according to claim 11, wherein the antigen reflects an incident light as a colored interference light when aligned in a film-like shape.

13. An antigen detecting agent according to claim 12, wherein the antigen detecting agent is amphiphilic.

14. An antigen detecting kit comprising:
an antigen detecting agent;
any one of a dish, a plate and a tube;
wherein the antigen detecting agent comprises a rod-shaped body and an antibody bonded to the rod-shaped body and a length of the rod-shaped body is 810 nm or shorter and specifically bonds to a target antigen and reflects the incident light as colored interference light when aligned in a film-like shape.

15. An antigen detecting apparatus comprising:
an antigen detecting agent;
means for adding a sample to the antigen detecting agent; and
means for measuring changes in a wavelength by the colored interference light of the antigen detecting agent aligned in a film-like shape to the target antigen;
wherein the antigen detecting agent comprises a rod-shaped body and a antibody bonded to the rod-shaped body and a length of the rod-shaped body is 810 nm or shorter and specifically bonds to a target antigen and reflects the incident light as colored interference light when aligned in a film-like shape.

16. An antigen detecting apparatus according to claim 15, wherein the antigen detecting agent is amphiphilic and the means for adding is an adding means in which the antigen detecting agent is added to an aqueous phase together with an oil phase so that the antigen detecting agent contacts the sample.

17. An antigen detecting apparatus comprising:
a biosensor having a rod-shaped body and an antibody bonded to the rod-shaped body and which specifically bonds to a target antigen in which an amphiphilic biosensor is aligned to one of a quartz oscillator and a surface acoustic wave (SAW) element in a film-like shape;
an oscillation circuit in which changes in mass or changes in viscoelasticity when a target antigen is bonded to the biosensor are oscillated as a frequency; and
a frequency counter in which a frequency oscillated from the oscillation circuit is measured;
wherein the antigen detecting agent comprises a rod-shaped body and an antibody bonded to the rod-shaped and a length of the rod-shaped body is 810 nm or shorter and which specifically bonds to a target antigen and reflects the incident light as colored interference light when aligned in a film-like shape.

18. An antigen detecting apparatus according to claim 17, wherein the antigen detecting agent is aligned in a monomolecular film-like shape to one of the quartz oscillator and the surface acoustic wave (SAW) element.

19. An antigen detecting apparatus according to claim 17, wherein an antigen detecting agent is adhered in a two layered monomolecular film-like shape to one of the quartz oscillator and the surface acoustic wave (SAW) element.

20. An antigen detecting method comprising:
a step for contacting a sample to an antigen detecting agent; and
a step for measuring changes in a wavelength by the colored interference light of the antigen detecting agent bonded to the target antigen;
wherein the antigen detecting agent comprises a rod-shaped body and an antibody bonded to the rod-shaped body and a length of the rod-shaped body is 810 nm or shorter and which specifically bonds to a target antigen and reflects the incident light as colored interference light when aligned in a film-like shape.
